(19) United States
(12) Patent Application Publication
(10) Pub. No.: US 2024/0302394 A1
(43) Pub. Date: Sep. 12, 2024

(54) System and Methods for Dipping Electrical Sensor for Measuring Properties of Molecules

(71) Applicant: HexagonFab, Cambridge (GB)

(72) Inventors: Amol Virendra Patil, Cambridge (GB); Paolo Romele, Cambridge (GB); Lukas James Vasadi, Cambridge, MA (US); Ruizhi Wang, Cambridge (GB)

(21) Appl. No.: 18/574,368

(22) PCT Filed: Jul. 1, 2022
(86) PCT No.: PCT/EP2022/068309

§ 371 Date: Dec. 27, 2023

(30) Foreign Application Priority Data
Jul. 1, 2021 (DE) 10 2021 117 034.6

(51) Int. Cl. G01N 35/00; B01L 3/00; G01N 33/543

(52) U.S. Cl. CPC G01N 35/0099; B01L 3/5085; G01N 33/5438; B01L 2300/0645; B01L 2300/0829

(57) ABSTRACT

The present invention relates to a system including a robotic actuator configured to engage an electrical sensor, a platform configured to retain a multi-well plate, and at least one processor configured to control the robotic actuator to dip the electrical sensor into wells filled with an analyst liquid, a sample liquid, a pre-detection liquid and a detection liquid. The present invention further relates to a corresponding method for determining the presence and/or concentration of a target molecule in a sample, for determining the binding kinetics and binding affinity of molecules and for determining the conformality structure.

### CROSS-REFERENCE TO RELATED APPLICATIONS

This application is the United States national phase of International Application No. PCT/EP2022/068309 filed Jul. 1, 2022, and claims priority to German Patent Application No. 10 2021 117 034.6 filed Jul. 1, 2021, the disclosures of which are hereby incorporated by reference in their entireties.

### BACKGROUND OF THE INVENTION

### Field of the Invention

The present invention relates to a system comprising a robotic actuator configured to engage an electrical sensor, a platform configured to retain a multi-well plate, and at least one processor configured to control the robotic actuator to dip the electrical sensor into wells filled with an analyte liquid, a sample liquid, a pre-detection liquid and a detection liquid. The present invention further relates to a corresponding method for determining the presence and/or concentration of a target molecule in a sample, for determining the binding kinetics and binding affinity of molecules and for determining the conformality structure.

### Description of Related Art

Properties of molecules, in particular interactions between molecules, are investigated in various use cases. For instance, for drug discovery and process monitoring, there is a need for low cost, rapid, accurate methods for analyzing interactions between molecules.

Examples are numerous and include studying binding kinetics of analyte molecules (e.g., representing future drugs) to target molecules; screening for the quantity and quality of a valuable protein molecule (e.g., monoclonal antibody) during various developmental and production steps; or mapping antigen epitopes to characterize and optimize antibody binding.

These types of measurements are currently performed on a variety of platforms, including High Performance Liquid Chromatography (HPLC) or Enzyme-Linked Immunosorbent Assays (ELISA) and increasingly various label-free assay technologies.

Well-established technologies such as HPLC and ELISA are limited in terms of the range of assays that can be run on these platforms. They also feature comparatively protracted times to result, offer relatively limited data compared to newer technologies and can only be operated by specially trained personnel in a centralized laboratory environment.

The limitations of traditional methods such as HPLC and ELISA have led to a move in recent years to label-free assay platforms. Label-free assays offer various benefits to the user, including real-time data read-out, quantitative data, and high sensitivity. There are several label-free platforms available, mostly based on the optical detection principles of Surface Plasmon Resonance (SPR) and BioLayer Interferometry (BLi). In SPR, sensors made of a thin gold film are used. Incoming light waves are absorbed and excite the electrons in the gold film into a state of collective oscillation, which is referred to as plasmon resonance. BLI is part of the general optical analysis technique called interferometry. A coherent light wave, i.e., light with only one well defined wavelength, is reflected from two surfaces separated by a small distance. The resulting diffraction pattern allows the determination of the distance between the two surfaces. An example of BLi is disclosed in US 2011268610 AA.

Optical sensing of molecule properties faces certain restrictions. For instance, the measurement equipment is comparably complex. An optical light path is required to and from a sensor head to a light source and detector of the sensor.

Thus, there is a clear need for advanced techniques of measuring properties of molecules. Specifically, there is a need for systems and methods that facilitate fast, reliable, quantitative, and reproducible measurements of the concentration, binding kinetics, affinity and conformality structure of molecules, in particular of biochemically relevant target molecules.

### SUMMARY OF THE INVENTION

The present invention addresses these needs and provides a system (100), comprising: a robotic actuator (102) configured to engage an electrical sensor (111), the electrical sensor (111) comprising a sensitive region (180) that can be functionalized using first molecules of a first type; a platform (170) configured to retain a multi-well plate (130), and at least one processor (801) configured to control (3005, 3020) the robotic actuator (102) to dip the electrical sensor (111) into wells (131-136) of the multi-well plate (130), wherein at least one of the wells (131-136) is filled with an analyte liquid comprising second molecules of a second type, wherein at least one further well (131-136) is filled with a sample liquid comprising a target molecule; wherein at least one further well (131-136) is filled with a pre-detection liquid comprising third molecules of a third type; wherein at least one further well (131-136) is filled with a detection liquid comprising fourth molecules of a fourth type; wherein the at least one processor (801) is configured to control (3010) the electrical sensor (111) to acquire one or more time sequences of data readouts (40) when the electrical sensor is dipped into the at least one well being filled with the detection liquid, wherein the at least one processor (801) is configured to determine (3030) at least one property of the target molecule or of molecules interacting with it based on at least some of the data readouts (40) of the one or more time sequences of data readouts (40). In a further aspect the present invention provides a corresponding method for determining the presence and/or concentration of a target molecule in a sample, comprising: (A) providing a robotic actuator (102) configured to engage an electrical sensor (111), the electrical sensor (111) comprising a sensitive region (180) that can be functionalized using first molecules of a first type, a platform (170) configured to retain a multi-well plate (130), and at least one processor (801) configured to control (3005, 3020) the robotic actuator (102) to dip the electrical sensor (111) into wells (131-136) of the multi-well plate (130), a multi-well plate comprising at least one of the wells (131-136) being filled with an analyte liquid comprising second molecules of a second type, at least one further well (131-136) being filled with a liquid sample comprising a target molecule; at least one further well (131-136) being filled with a pre-detection liquid comprising third molecules of a third type; and at least one further well (131-136) being filled with a detection liquid comprising fourth molecules of a fourth type; (B) dipping said electrical sensor into at least one well (131-136) filled with an analyte liquid comprising second molecules of a second type, dipping said electrical sensor into at least one further well (131-136) filled with a sample liquid comprising a target molecule; dipping said electrical sensor into at least one further well (131-136) filled with a pre-detection liquid comprising third molecules of a third type; and dipping said electrical sensor into at least one further well (131-136) filled with a detection liquid comprising fourth molecules of a fourth type; (C) measuring an electrical signal in dependence of the electrochemical condition of the fourth molecules, indicating the presence and/or concentration of the target molecule.

The present inventors have surprisingly found that the above-mentioned system and method is capable of accurately measuring properties such as concentration, affinity, binding kinetics and conformality with minimal impact by the buffer composition, while providing high sensitivity and large dynamic range. Compared to traditional methods, such as ELISA the integrated automation results in minimal hands-on time. Furthermore, the system and methods provide an increased dynamic range, i.e. the range between the smallest and largest measurable concentrations is significantly augmented. Compared to prior art methods such as SPR or BLI, the approach according to the present invention has, inter alia, the advantage that it does not require lengthy baseline measurements and is insensitive to the measurement buffer composition, i.e. measurements can be performed in purified solution, media or biological sample fluids (e.g. blood, sweat, urine) without interference from the background.

In a preferred embodiment of the system according to the invention the at least one processor (801) is configured to determine the at least one property based on a timing of said controlling of the robotic actuator (102) to dip the electrical sensor (111) into one or more wells (131-136) of the multi-well plate (130).

In a further preferred embodiment of the system according to the invention the at least one of the wells (131-136) being filled with an analyte liquid comprising second molecules of a second type is not present in the system.

In a further embodiment of the system the at least one processor (801) is configured to control the robotic actuator (102) and the electrical sensor (111) in accordance with a predefined measurement script (185) and the predefined measurement script (185) is parameterized based on at least one parameter, a value of the at least one parameter being set based on at least one of the data readouts (40) of the one or more time sequences of data readouts (40).

In a particularly preferred embodiment the predefined measurement script (185) defines a detection phase, the at least one processor (801) is configured to control the robotic actuator (102) to dip the electrical sensor (111) into the at least one well of the multi-well plate filled with the detection liquid during the detection phase, the at least one property comprises a concentration, the concentration being determined using a regression analysis of a predefined curve (45) to multiple data readouts (40) of the data readouts (40) of a time sequence of data readouts (40) acquired during the detection phase (59), the at least one parameter of the predefined measurement script comprises a time gate (51, 52) for the regression analysis, and a value of the time gate (51, 52) is set based on a timing of said controlling of the robotic actuator (102) to dip the electrical sensor (111) into the at least one well (131-136) of the multi-well plate (130) filled with the detection liquid.

In yet another embodiment of the system the robotic actuator (102) is configured to engage a further electrical sensor (112) so that the electrical sensor (111) and the further electrical sensor (112) are arranged at an offset which corresponds to an offset between wells (133, 134) of the multi-well plate (130), the at least one processor (801) is configured to control the further electrical sensor (112) to acquire one or more further time sequences of further data readouts when the further electrical sensor (112) is dipped into the at least one well being filled with the detection liquid and the at least one processor (801) is configured to determine the at least one property using a reference baseline obtained from at least some of the further data readouts.

In a further embodiment of the system the at least one processor (801) is configured to control the robotic actuator (102) and the electrical sensor (111) in accordance with a predefined measurement script (185), the predefined measurement script (185) defines a preparation phase (57), the at least one processor (801) is configured to control the robotic actuator (102) to dip the electrical sensor (111) into a second one of the one or more wells (131-136) of the multi-well plate filled with a liquid comprising the first molecules during the preparation phase (57), and the at least one processor is configured to not dip the further electrical sensor (112) into any well (131-136) of the multi-well plate (130) filled with the liquid comprising the first molecules during the preparation phase (57).

In a further embodiment of the system the at least one processor (801) is configured to control at least one of the robotic actuator (102) or a motor attached to the platform (170) to relative move the electrical sensor (111) with respect to and within the at least one of the one or more wells (131-136) when the electrical sensor (111) is dipped into each one of the at least one of the one or more wells (131-136).

In particularly preferred embodiment the present invention relates to a system as defined herein above, wherein the at least one property comprises at least one of
(i) binding kinetics of a binding between the second molecules and the target molecules, or of the third molecules and the second molecules or target molecules;
(ii) a binding affinity of the binding between the second molecules and the target molecules or of the third molecules and the second molecules or target molecules,
(iii) a concentration of the target molecules in the sample liquid, or
   (iv) a conformality structure of the target molecules.

In a preferred embodiment of the method as defined above the dipping steps are performed in the following order: (1) dipping said electrical sensor into at least one well (131-136) filled with an analyte liquid comprising second molecules, (2) dipping said electrical sensor into at least one further well (131-136) filled with a sample liquid comprising a target molecule, (3) dipping said electrical sensor into at least one further well (131-136) filled with a pre-detection liquid comprising third molecules of a third type; and (4) dipping said electrical sensor into at least one further well (131-136) filled with a detection liquid comprising fourth molecules of a fourth type further embodiment.

In a further embodiment the above mentioned method is for determining the binding kinetics of a binding between the second molecules and the target molecules, or of the third molecules and the second molecules or target molecules and comprises a repetition of step (B) and (C) after a predefined period of time, preferably after 30 sec, 1, 2, 3, 4, 5, 10, 20, 30, 60 min to 5 h, wherein said repetition is a 1×, 2×, 3×, 4×, 5× or multiple repetition.

In a further embodiment the above mentioned method is for determining the binding affinity of the binding between the second molecules and the target molecules or of the third molecules and the second molecules or target molecules and comprises a repetition of step (B) and (C) wherein after each execution or repetition of steps (B) and (C) the dipping of said electrical sensor into at least one further well (131-136) is performed in a well which comprises an increased or decreased concentration of the pre-detection liquid comprising third molecules of a third type in comparison to the previously used well.

It is particularly preferred that said concentration is increased or decreased by a factor 2, 3, 4, 5, 6, 7, 8, 9, 10 or more in comparison to the previously used well.

In a further particularly preferred embodiment of said method, after each execution or repetition of steps (B) and (C) said electrical sensor is in step (D) dipped into at least one well (131-136) filled with a dissociation liquid capable of dissociating the bond between the second molecule of a second type and the third molecule of a third type.

In yet another particularly preferred embodiment after each dipping step the sensor is additionally dipped into at least one well (131-136) filled with a rinsing liquid.

In a further embodiment the above mentioned method is for determining the conformality structure of the target molecules, comprising a repetition of step (B) and (C) wherein after each execution or repetition of steps (B) and (C) the dipping of said electrical sensor into at least one further well (131-136) is performed in a well which comprises a liquid with a pH differing from the pH of the liquid in the previously used well.

According to specific embodiments of the system or method according to the present invention said second molecule is an antibody or a specifically binding non-immunoglobulin.

According to further specific embodiments of the system or method according to the present invention said third molecule is an antibody or specifically binding non-immunoglobulin which is conjugated to an enzymatic activity, preferably HRP, alkaline phosphatase or glucose oxidase.

According to further specific embodiments of the system or method according to the present invention said third molecule is an antibody or specifically binding non-immunoglobulin, which is conjugated to an enzymatic activity, preferably HRP, alkaline phosphatase or glucose oxidase.

According to further specific embodiments of the system or method according to the present invention said fourth molecule is an electrochemically sensitive molecule whose electrochemical condition can be changed by the enzymatic activity.

According to preferred embodiments said method as defined herein above is performed with the system according to the present invention.

### BRIEF DESCRIPTION OF THE DRAWINGS

FIG. 1 schematically illustrates a system for performing measurements on molecules according to various examples.

FIG. 2 schematically illustrates positioning an electrical sensor attached to a robotic actuator with respect to wells of a MWP according to various examples.

FIG. 3 schematically illustrates the electrical sensor being retracted into the well of the MWP according to various examples.

FIG. 4 schematically illustrates the electrical sensor being immersed in a fluid in the well of the MWP according to various examples.

FIG. 5 is a flowchart of a method according to various examples.

FIG. 6 schematically illustrates a time sequence of data readouts according to various examples.

FIG. 7 shows results of an experiment performed with 3 sensors to measure the concentration differences in the analyte.

### DETAILED DESCRIPTION OF THE INVENTION

Although the present invention will be described with respect to particular embodiments, this description is not to be construed in a limiting sense.

Before describing in detail exemplary embodiments of the present invention, definitions important for understanding the present invention are given.

It is generally to be understood that the features mentioned herein may be used not only in the respective combinations indicated, but also in other combinations or in isolation without departing from the scope of the invention

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” or “essentially consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “(i)”, “(ii)”, “(iii)” or “(A)”, “(B)”, “(C)”, “(D)”, or “first”, “second”, “third” etc. and the like in the description or in the claims, are used for distinguishing between similar elements and not necessarily for describing a sequential or chronological order, unless otherwise indicated. It is to be understood that the terms so used are interchangeable under appropriate circumstances and that the embodiments of the invention described herein are capable of operation in other sequences than described or illustrated herein. In case the terms relate to steps of a method or use there is no time or time interval coherence between the steps, i. e. the steps may be carried out simultaneously or there may be time intervals of seconds, minutes, hours, days, weeks etc. between such steps, unless otherwise indicated.

It is to be understood that this invention is not limited to the particular system, elements, methodology, protocols, reagents etc. described herein as these may vary. It is also to be understood that the terminology used herein is for the purpose of describing particular embodiments only and is not intended to limit the scope of the present invention that will be limited only by the appended claims. Unless defined otherwise, all technical and scientific terms used herein have the same meanings as commonly understood by one of ordinary skill in the art.

Further, some examples of the present disclosure generally provide for a plurality of circuits or other electrical devices. All references to the circuits and other electrical devices and the functionality provided by each are not intended to be limited to encompassing only what is illustrated and described herein. While particular labels may be assigned to the various circuits or other electrical devices disclosed, such labels are not intended to limit the scope of operation for the circuits and the other electrical devices. Such circuits and other electrical devices may be combined with each other and/or separated in any manner based on the particular type of electrical implementation that is desired. It is recognized that any circuit or other electrical device disclosed herein may include any number of microcontrollers, a graphics processor unit (GPU), integrated circuits, memory devices (e.g., FLASH, random access memory (RAM), read only memory (ROM), electrically programmable read only memory (EPROM), electrically erasable programmable read only memory (EEPROM), or other suitable variants thereof), and software which co-act with one another to perform operation(s) disclosed herein. In addition, any one or more of the electrical devices may be configured to execute a program code that is embodied in a non-transitory computer readable medium programmed to perform any number of the functions as disclosed.

In the following, techniques of automating measurements for determining properties of molecules of one or more types will be described. Respective measurement protocols defining a sequence of measurement phases and associated actions are disclosed. For instance, binding properties can be between molecules of a first type and molecules of a second type can be determined. Binding properties between target molecules and analyte molecules can be determined. According to a specific embodiment, the present invention envisages that there is no binding between a molecule of a first type and molecules of a third type.

Such measurements are implemented using an electrical sensor. The electrical sensor may be functionalized. More specifically, a sensitive surface of the electrical sensor may be functionalized using sensor-side molecules.

According to various examples, a robotic actuator is employed to automate the measurements. Specifically, the—possibly functionalized-electrical sensor can be engaged by the robotic actuator and then dipped into one or more wells. The sensor, and a sensitive region of the sensor, can thus be immersed in a liquid. The liquid can be a solution of molecules of a certain type. Molecules could also be included as a dispersion.

By dipping the electrical sensor into liquids including the molecules, it is possible to reliably bring the molecules into contact with a sensitive region of the electrical sensor. Further, a timing of exposure of the sensitive surface to the molecules can be precisely captured based on the dipping process. Complex measurement protocols can be automated, e.g., using a multi-well plate including multiple wells including different liquids. Different phases of a measurement protocol can be implemented. One or more properties can be quantified accurately.

According to various examples, a system according to the present invention includes a robotic actuator. The robotic actuator is configured to engage an electrical sensor. The electrical sensor includes a sensitive surface. The sensitive surface can be functionalized using first molecules of a first type. The system also includes a platform that is configured to retain a multi-well plate (MWP). Also, the system includes at least one processor. The at least one processor is configured to control the robotic actuator to dip the electrical sensor into wells of the MWP. The at least one of the wells is filled with an analyte liquid including second molecules of a second type. The at least one further well is filled with a sample liquid comprising a target molecule. At least one further well is filled with a pre-detection liquid comprising third molecules of a third type and at least one further is filled with a detection liquid comprising fourth molecules of a fourth type. The at least one processor is configured to control the electrical sensor to acquire one or more time sequences of data readouts when the electrical sensor is dipped into the at least one well being filled with the detection liquid. The at least one processor is further configured to determine at least one property of at least one the target molecule or of molecules interacting with it based on at least some of the data readouts of the one or more time sequences of data readouts.

In certain additional embodiment, the invention further relates to computer-implemented methods. Such methods include steps for controlling a robotic actuator.

According to the present invention, the robotic actuator engages an electrical sensor. The electrical sensor can be functionalized using first molecules of a first type. The robotic actuator is controlled to dip the electrical sensor into one or more wells of a multi-well plate. At least one of wells is filled with an analyte liquid including second molecules of a second type. The at least one further well is filled with a sample liquid comprising a target molecule. At least one further well is filled with a pre-detection liquid comprising third molecules of a third type and at least one further is filled with a detection liquid comprising fourth molecules of a fourth type. The computer-implemented method also includes controlling the electrical sensor to acquire one or more time sequences of data readouts when the electrical sensor is dipped into each one of the wells. The computer-implemented method also includes determining at least one property of at least the target molecule or of molecules interacting with it based on at least some of the data readouts.

A computer program or a computer program product or a computer-readable storage medium includes program code. The program code can be loaded and executed by at least one processor. Upon loading and executing the program code, the at least one processor performs a method. The method includes controlling a robotic actuator. The robotic actuator engages in electrical sensor. The electrical sensor can be functionalized using first molecules of a first type. The robotic actuator is controlled to dip the electrical sensor into one or more wells of a multi-well plate. At least one of the one or more wells is filled with an analyte liquid including second molecules of a second type. The at least one further well is filled with a sample liquid comprising a target molecule. At least one further well is filled with a pre-detection liquid comprising third molecules of a third type and at least one further is filled with a detection liquid comprising fourth molecules of a fourth type.

The computer-implemented method also includes controlling the electrical sensor to acquire one or more time sequences of data readouts when the electrical sensor is dipped into each one of the one or more wells. The computer-implemented method also includes determining at least one property of the target molecule or of molecules interacting with it.

In the following, embodiments of the invention will be described in detail with reference to the accompanying drawings. It is to be understood that the following description of embodiments is not to be taken in a limiting sense. The scope of the invention is not intended to be limited by the embodiments described hereinafter or by the drawings, which are taken to be illustrative only.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Hereinafter, techniques of performing a measurement of one or more properties of one or molecules will be described. Specifically, properties of analyte molecules could be determined.

Such measurements may facilitate various use cases. Surface science can be facilitated where properties of a surface formed by certain molecules is investigated. For instance, proteins or small molecules for research of pharmaceutical drugs may be facilitated. Ribonucleic acid (RNA) or Deoxyribonucleic acid (DNA) can be investigated. Molecule-binding assays could be implemented. Antibody-antigen kinetics could be measured. The quantity and/or quality of a protein molecule such as a monoclonal antibody could be measured. Antigen epitopes could be mapped to characterize and optimize antibody binding.

As a general rule, different kinds of measurements can be implemented and, along with different kinds of measurements, different properties can be determined. For instance, it would be possible to determine a property of a binding/adsorption between target molecules and analyte molecules. Binding parameters may include binding kinetics or a binding affinity. The binding kinetics can specify how fast the target and analyte molecules bind. Binding affinity can specify a strength of the binding. Another option would be to determine a concentration of the analyte molecules in an analyte liquid. Yet another option be to determine a conformality structure of analyte molecules.

Electrical sensing is employed. Specifically, an electrical sensor is used. This means, that an electrical sensor signal is used to determine one or more properties as identified above. For instance, a time-dependency of the electrical sensor signal could be used to determine the binding kinetics.

As a general rule, according to the various examples disclosed herein, various kinds and types of electrical sensors can be used.

Some examples are summarized in TAB. 1.

TABLE 1
Various options for electrical sensors used for measurements

| Example | Brief description | Example details |
|---|---|---|
| I | Field-effect | One example would be a field-effect transistor that includes |
| | transistor (FET) | a sensitive region. Here, electrical fields can be |
| | | created/changed due to presence of molecules at or adjacent |
| | | to the sensitive region. The sensitive region thus forms a gate |
| | | contact of the FET. The electrical fields, in turn, can then |
| | | affect mobility of charge carries in a channel between source |
| | | and drain contacts below the gate contact. Thus, a current |
| | | through the FET can be measured and one or more |
| | | properties of molecules can be determined based on the |
| | | current. |
| | | There are, furthermore, various options available to |
| | | implement the sensitive region. |
| | | In some examples, the sensitive region comprises graphene |
| | | as an electrical field sensitive layer. Thereby, a material with |
| | | a large change in electrical properties (e.g., resistance) in |
| | | response to an applied electric field can be provided as the |
| | | electric field sensitive layer thus providing a high sensitivity |
| | | to electric fields caused by molecules arranged adjacent to |
| | | the sensitive region. |
| | | In some examples, the sensitive region includes one or more |
| | | of nanowires, nanotubes, and a two-dimensional material as |
| | | an electrical field sensitive layer. Materials which can form |
| | | suitable nanowires include, for example, silicon, gallium |
| | | arsenide (GaAs), indium arsenide (InAs) and Galium Nitride |
| | | (GaN). Materials which can form suitable nanotubes include, |
| | | for example, carbon and transition metal dichalcogenide. |
| | | Materials which can form suitable two-dimensional materials |
| | | include, for example, graphene, phosphorene, silicene, |
| | | germanene and transition metal dichalcogenides. Thereby, |
| | | through the provision of low dimensional materials, a |
| | | sensitive region which has a high sensitivity to electric fields |
| | | can be provided. |
| | | In some examples, the electric field sensitive layer comprises |
| | | a bulk semiconductor as an electrical field sensitive layer. |
| | | Thereby, inexpensive and readily available materials with |
| | | established processing techniques can be used for the |
| | | electric field sensitive layer. Examples of suitable |
| | | semiconductors include: silicon, germanium, gallium arsenide |
| | | (GaAs), an alloy of silicon and germanium, indium phosphide |
| | | and gallium nitride. |
| | | An example implementation of the FET is disclosed in WO |
| | | 2020/099890. The respective disclosure is incorporated |
| | | herein by reference. |
| II | Micro-electrical | A MEMS-based sensor can, e.g., include a mechanical |
| | mechanical | oscillator. The sensitive region could then be implemented by |
| | system (MEMS) | an oscillating mass of the mechanical oscillator. Molecules |
| | | binding to or arranged adjacent to the oscillating mass can |
| | | affect a resonance frequency of the oscillating mass. Thus, a |
| | | frequency-shift of the resonance frequency can be used as |
| | | electrical signal to determine one or more properties as |
| | | identified above. |
| III | Electrochemical | An electrochemical cell including a working, a reference or a |
| | Sensor | working, a reference and a counter electrode. Molecules |
| | | binding on the working electrode alter the properties of the |
| | | electrochemical cell. This can be read out using a multitude |
| | | of methods, e.g., linear voltammetry, cyclic voltammetry, |
| | | amperometry, potentiometry, electrical impedance |
| | | spectroscopy. Preferably, the electrochemical sensor |
| | | measures an electrical current flow. |

According to various examples, the sensitive region can be functionalized. According to various examples, it would be possible that the sensitive region is pre-functionalized, i.e., sensor-side molecules are already attached to the sensitive region. It would also be possible that the sensitive region is being functionalized using techniques disclosed herein in a preparation phase of the measurement protocol. In the preparation phase, the electrical sensor is dipped into a liquid of sensor-side molecules. The sensor-side molecules then can adhere to the sensitive region, to thereby functionalize the electrical sensor. To facilitate such binding, the sensitive region can include an adhesion layer to support adhesion of the sensor-side molecules. This could be, e.g., a hexagonal boron nitride layer.

According to various examples, the electrical sensor is dipped into one or more wells, e.g., of a MWP, to bring a sensitive region of the electrical sensor into contact with respective liquid-side molecules included in a respective liquid, e.g., in solution.

Such dipping can be automated using a robotic actuator. The robotic actuator can engage one or more electrical sensors, cf. TAB. 1. Then, an MWP can be arranged on a platform and a processor can be configured to control the robotic actuator to dip the one or more electrical sensors into one or more wells of the MWP.

In particular, it would be possible to use a measurement script. The measurement script can specify different actions of the robotic actuator. Thereby, the measurement protocol can be implemented. For instance, the timing of the dipping, e.g., start time and duration, can be appropriately set. A sequence of wells of the MWP into which an electrical sensor is dipped can be set. Sampling durations during which data readouts are performed can be defined. The electrical sensor can be controlled to acquire one or more time sequences of data readouts in accordance with the predefined measurement script.

Then, based on such data readouts, the at least one processor can be configured to determine at least one property of molecules of one or more types. For instance, a binding property between target molecules and analyte molecules may be measured. It would be possible to measure a property of analyte molecules, e.g., activity, concentration, etc. The analyte molecules can be implemented by the sensor-side molecules or the liquid-side molecules.

Specifically, according to various examples, it would be possible to take into account the measurement script when determining the at least one property. Here, it would be possible that, based on the measurement script, data readouts or time sequences of data readouts are associated with certain liquids to which the electrical sensor is exposed when being dipped into respective wells. Specifically, it would be possible that based on the measurement script, data readouts or time sequences of data readouts are associated with different phases of a measurement protocol, e.g., preparation phase, an association phase, an optional calibration phase, a pre-detection phase, a detection phase or a dissociation phase. Thus, it would be possible that the at least one property is determined based on a timing of said controlling of the robotic actuator to dip the electrical sensor into the one or more wells of the MWP. This timing can be defined by the measurement protocol.

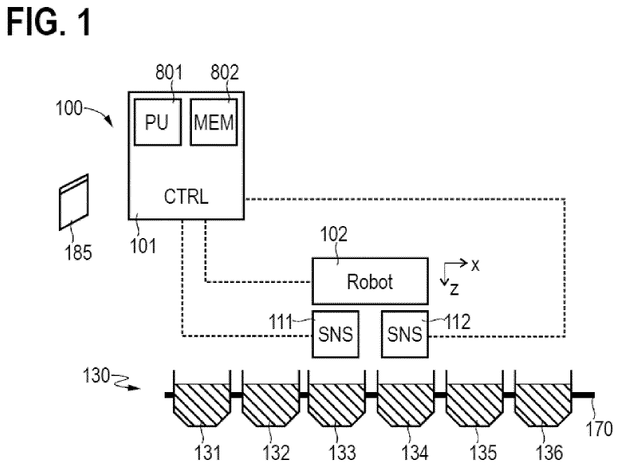

FIG. 1 schematically illustrates a system 100 according to various examples. The system 100 is configured for performing measurements to determine one or more properties of molecules. Specifically, properties of analyte molecules can be determined. The analyte molecules can be in a liquid, i.e., liquid-side molecules; but it would also be possible that the analyte molecules are attached to a sensor surface, i.e., sensor side molecules. An electrical sensor 111 that is used for the measurements can be functionalized using sensor-side molecules.

To determine properties of the analyte molecules, it would be possible to use target molecules. One or more properties of the target molecules may be known. Then, based on prior knowledge, it is possible to determine the one or more properties of the analyte molecules. The target molecules (analyte molecules) can be implemented by the sensor-side surface molecules (liquid-side molecules); or vice versa.

In some experiments, an antibody is immobilized on the sensor (i.e., as analyte molecule) and the target protein is measured in solution. It could also be implemented such that the target protein is immobilized on the sensor and the antibody is in solution as liquid-side molecule.

The system 100 includes a control device 101 that can communicate with a robotic actuator 102 and electrical sensors 111, 112. The electrical sensors 111, 112 are both engaged by the robotic actuator 102. In particular, the electrical sensors 111, 112 could be releasably engaged, so that they can be replaced in between subsequent measurements.

The control device 101 can include at least one processor 801 (labelled “PU” in FIG. 1, processor unit) and a memory 802. The at least one processor 801 could be implemented by a general-purpose processing unit, and an application-specific integrated circuit, or a field-controlled gated array, to give just a few examples. The at least one processor 801 could load program code from the memory 802 and execute the program code. Upon loading and executing the program code, the at least one processor 801 can perform techniques as described herein, e.g., control the robotic actuator to move, e.g., to position the electrical sensors 111, 112 in or above certain wells 131-136 of a MWP 130 that is arranged on a platform 170 or to dip the electrical sensors 111, 112 into respective wells 131-136, determine one or more properties of analyte molecules, read a predefined measurement script 185 and control the robotic actuator 102 and/or the electrical sensor 111, 112 based on the predefined measurement script 185, etc.

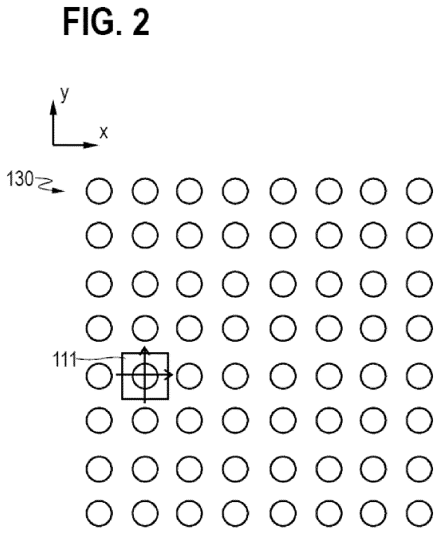

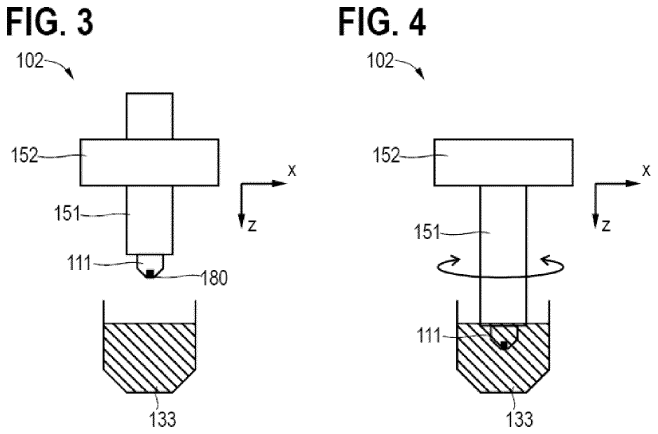

The robotic actuator 102 can be controlled by the control device 101 to move the electrical sensors 111, 112. Depending on the structural implementation of the robotic actuator 102, different degrees of freedom of movement of the robotic actuator 102 are possible. For instance, a translational movement, e.g., along all three spatial axes, would be possible. Here, a lateral movement (along X axis and Y axis) could be used to select a specific well of a multi-well plate (this is illustrated in FIG. 2, where a 2-D array of wells of the MWP 130 is illustrated using the circles), and a depth movement (along the Z axis) could be used to dip the electrical sensor into a respective well 131-136 of the MWP 130 (this is illustrated in FIG. 3 which shows a retracted position and in FIG. 4 which shows a dipped position; here a piston 151 of the robotic actuator 102 moves with respect to a base plate 152, to implement the depth movement). It would also be possible that the robotic actuator 102 can perform a rotational movement, e.g., rotation around the Z axis (cf. FIG. 4). This can be helpful to stir the electrical sensors 111, 112 when dipped in a respective liquid in the wells 131-136.

In the example illustrated in FIG. 1, the robotic actuator 102 could perform a movement along the Z axis; this would result in dipping the electrical sensor 111 into the well 133 and the electrical sensor 112 into the well 134. Thus, because the electrical sensor 111 and the electrical sensor 112 are arranged at an offset with respect to each other (when engaged by the robotic actuator 102) that corresponds to the offset between the wells 131-136 of the MWP, it is possible to jointly dip multiple electrical sensors 111, 112 into separate wells with a single translational movement of the robotic actuator 102.

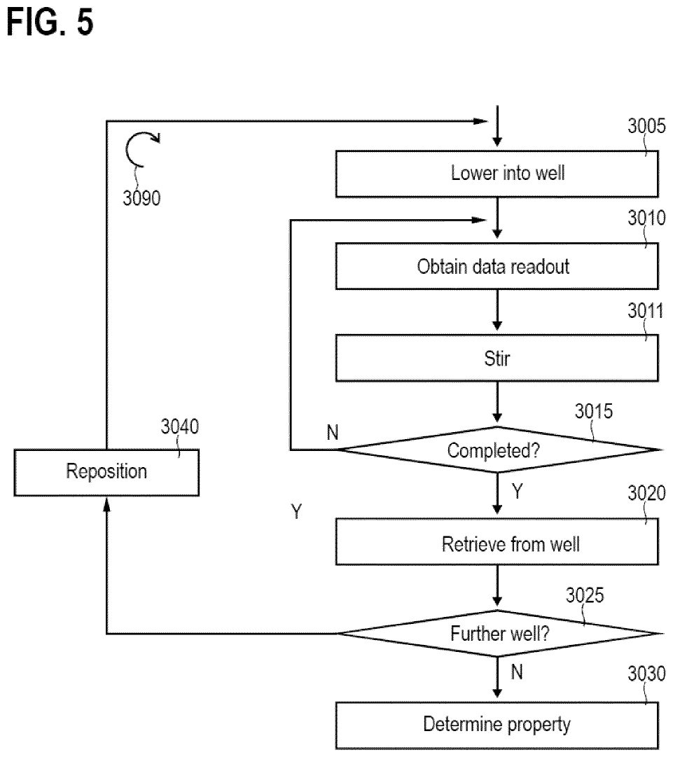

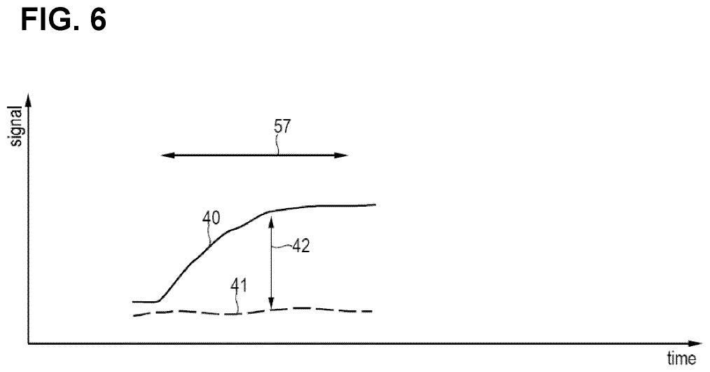

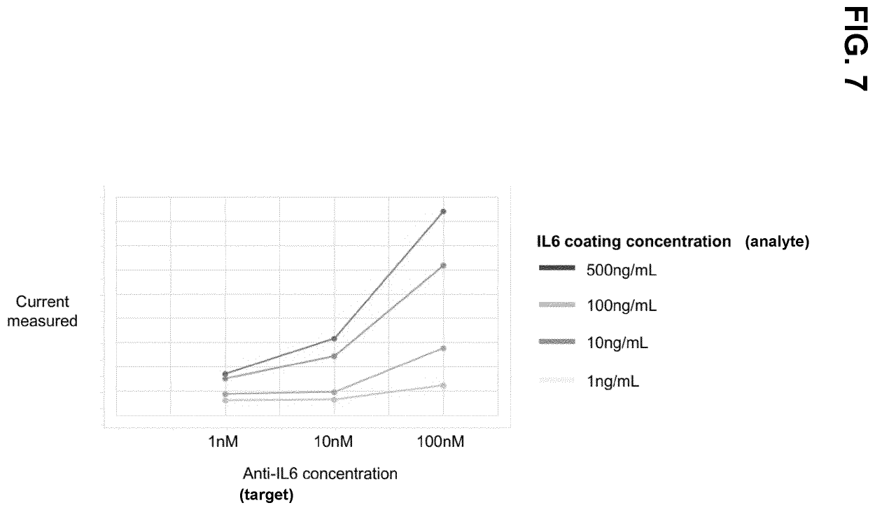

FIG. 5 is a flowchart of a method according the various examples. The method of FIG. 5 could be executed by at least one processor of a measurement system. For instance, the method of FIG. 5 could be executed by at least one processor 801 of the control device 101 of the system 100 of FIG. 1.

At box 3005, an electrical sensor lowered into a well of an MWP. This can include controlling a robotic actuator to lower the electrical sensor into the well (Z-movement). For instance, respective digital control instructions may be provided to the robotic actuator. Also, analog control, e.g., using voltage levels, would be possible, depending on the structural implementation of the robotic actuator.

Then, at box 3010, a data readout or multiple data readouts can be obtained. Box 3010, accordingly, can include controlling the electrical sensor to acquire a data readout. The data readout is representative of an electrical observable sensed by a sensitive region of the electrical sensor when the electrical sensor is immersed in a fluid in the well, e.g., frequency shift for MEMS (cf. TAB. 1, example II) or current for FETs (cf. TAB. 1, example I).

At optional box 3011, it would be possible that—while the sensitive region of the electrical sensor remains immersed in the fluid included in the well into which the electrical sensor has been lowered at box 3055—the robotic actuator is controlled to move the electrical sensor within and with respect to the respective well. For instance, this could include a rotation to stir (cf. FIG. 4) or a shaking movement. Thereby, local concentration gradients of molecules in solution in the respective liquid are avoided.

Alternatively or additionally to controlling the robotic actuator, it would also be possible to control a motor attached to a platform on which the MWP is mounted the electrical sensor to move the platform against the electrical sensor.

At box 3015 it can be checked whether one or more further data readouts are required while the electrical sensor is immersed in a fluid in the well. In the affirmative, further data readouts are obtained by one or more further iterations of box 3010. Thereby, a time sequence of data readouts is obtained, when and while the electrical sensor is dipped into the well.

On the other hand, if it is judged at box 3015 that a sampling duration during which the time sequence of data readouts is obtained by multiple iterations of box 3010 is completed, the method commences at box 3020.

At box 3020, the electrical sensor is retrieved from the well. Box 3020 can include controlling the robotic actuator to retrieve the electrical sensor from the well (Z-movement).

Box 3005 and box 3020 thus implement dipping the electrical sensor into a respective well of the MWP. A time offset between executing box 3005 and box 3020 defines a dwell time of the electrical sensor in the respective well.

At box 3025, it is checked whether the electrical sensor is to be dipped into a further well. In the affirmative, boxes 3005, 3010, 3015, and 3020 are executed in a respective iteration 3090, after the robotic actuator has been controlled to re-position to select another well (cf. FIG. 2; X-Y-movement), at box 3040.

Thus, by executing multiple iterations 3090, it is possible that the processor controls the robotic actuator to dip the electrical sensor into multiple wells of the MWP.

Once all iteration 3090 have been completed, i.e., time sequences of data readouts have been required for all required wells, the method then commences at box 3030. Here, one or more properties associated with molecules are determined based on the time sequence(s) of data readouts for each iteration 3090.

As a general rule, techniques for determining properties of the analyte molecules such as, e.g., binding properties to target molecules based on the time sequences of data readouts are known in the art and these techniques can be used herein. As a general rule, the analyte molecules may be attached to a sensor surface, i.e., may be sensor-side molecules. The analyte molecules could also be included in a liquid, i.e., liquid-side molecules.

For instance, it would be possible to determine an absolute signal level of the electrical signal captured by the data readouts. This absolute signal level could be compared against a reference. Thereby, it would be possible to determine a concentration. For instance, larger absolute signal levels can correspond to higher concentrations.

Another option would be to determine a change rate of the signal level. The change rate could be indicative of a binding kinetics. For instance, larger change rates could be indicative of faster binding.

These are only some options and other options are possible.

According to various examples, it is possible that the at least one property is determined at box 3030 based on the timing of said controlling of the robotic actuator to dip the electrical sensor into one or more wells of the MWP.

For instance, if a start time (defined by the timing of executing box 3005) or a stop time (defined by the timing of executing box 3020) of dipping the electrical sensor into a given well is known, this timing can be used in order to discriminate between data readouts acquired by the electrical sensor while the electrical sensor is being dipped into the respective well or before and after the electrical sensor is being dipped into the respective well. In particular, for low signal-to-noise ratios, it can be helpful to be able to judge between data readouts associated with only noise (i.e., before and after the electrical sensor is being dipped into a respective well) and data readouts associated with the signal (i.e., while the electrical sensor is being dipped into a respective well). This is, in particular, helpful if compared to manual techniques where a liquid including molecules is being pipetted onto the sensitive surface. Here, due to the manual process and surface tension preventing immediate wetting of the sensitive region, it can sometimes be difficult to judge when the sensitive region comes into contact with the (analyte) molecules.

The various iterations 3090 can be associated with or define different phases of a measurement protocol. Some of these phases are summarized in TAB. 2 below.

TABLE 2
Phases of a measurement protocol. The different phases can be implemented using a
measurement script. The measurement script can include (e.g., parameterized) control
instructions for controlling the robotic actuator and/or the electrical sensor to
implement such phases by dipping the electrical sensor into the respective well.

| Phase | Example description |
|---|---|
| Optional | In the optional calibration phase, the robotic actuator can be controlled to |
| calibration | dip the electrical sensor into a particular well that is filled with a reference |
| phase | liquid. For instance, the reference liquid could be water or a buffer solution. |
| | The reference liquid may not include a certain type of molecules, e.g., may |
| | not include analyte molecules. |
| | The reference liquid can have predefined properties so that the electrical |
| | signal output by the electrical sensor and captured by the respective time |
| | sequence of data readouts can serve as a reference. |
| | This phase may in certain embodiments not be performed. |
| Dissociation | A reference liquid can be used to (re-)transition the electrical sensor into a |
| phase | predefined state. For instance, if the sensitive region of the electrical sensor |
| | has been functionalized using sensor-side molecules, it would be possible |
| | that during the dissociation phase the sensor-side molecules are released |
| | from the sensitive surface, e.g., to go into solution in the reference liquid. |
| | During the dissociation phase it would also be possible to release liquid-side |
| | molecules that have been attached to sensor-side molecules. |
| Association | In the association phase, the robotic actuator can be controlled to dip the |
| phase | electrical sensor - that may or may not be functionalized using sensor-side |
| | molecules, see preparation phase - into a particular well that is filled with |
| | an analyte liquid. The analyte liquid includes the liquid-side molecules of a |
| | certain type. Then, the electrical signal of the electrical sensor is captured |
| | by the respective time sequence of data readouts can be indicative of one |
| | or more properties of the sensor-side molecules and/or the liquid-side |
| | molecules. |
| | For instance, the liquid-side molecules may come into contact with the |
| | sensitive region. For instance, the sensitive region may have been |
| | functionalized using sensor-side molecules and the liquid-side molecules |
| | may chemically bind to the sensor-side molecules. |
| | In certain embodiments the association phase with an analyte liquid |
| | comprising second molecules of a second type may be skipped. Such a |
| | skipping may take place in situations in which the preparation phase |
| | comprises a functionalization of the sensor with a molecule of a first type, |
| | which is equivalent to a molecule of second type. For example, said |
| | molecule of a first type being equivalent to a molecule of a second type may |
| | be Protein A, which could be both, a molecule of a first type and a molecule |
| | of a second type. |
| | In further embodiments, the association phase may comprise a step in |
| | which the robotic actuator can be controlled to dip the electrical sensor into |
| | a particular well that is filled with a sample liquid comprising a target |
| | molecule. Said target molecule may accordingly come into contact with the |
| | said second molecule of a second type and be associated to it. |
| Preparation | In the preparation phase, the robotic actuator can be controlled to dip the |
| phase | electrical sensor into a particular well that is filled with a liquid including |
| | sensor-side molecules. Then, the electrical sensor can be functionalized |
| | when the sensor-side molecules bind to the sensitive region of the electrical |
| | sensor. Thus, the preparation phase may precede the association phase. |
| Pre-detection | In the Pre-detection phase, the robotic actuator can be controlled to dip the |
| phase | electrical sensor into a particular well that is filled with a pre-detection |
| | liquid comprising third molecules of a third type. The pre-detection phase |
| | precedes the detection phase. |
| Detection | In the Detection phase, the robotic actuator can be controlled to dip the |
| phase | electrical sensor into a particular well that is filled with a detection liquid |
| | comprising fourth molecules of a fourth type. The presence of molecules of |
| | the third type and of molecules of the fourth type allow for the production |
| | of electrical current flow which can immediately be detected at the sensor. |

For instance, a concrete implementation of a measurement protocol could be as follows: (i) Well 1: Optional calibration phase-buffer solution, 5 min-establish baseline; (ii) Well 2: preparation phase-sensor-side molecules 5 min-adsorb sensor-side molecules onto surface of sensitive region (iii) Well 3: Optional calibration phase-buffer solution, 5 min-Confirm that sensor-side molecules are stuck onto the surface and do not detach; (iv) Well 4-association phase, liquid-side molecule 5 min-Binding of liquid-side molecules to sensor-side molecules; (v) Well 5-pre-detection phase, pre-detection liquid containing electrochemically active molecules 5 min-interaction of sensor with sensor-side molecule and liquid side molecule with; (vi) Well 6—detection phase, detection liquid 1 min-reaction between electrochemically active molecule and detection liquid; (vii) Well 7: Dissociation phase-buffer solution, 5 min-Monitor how quickly the liquid-side molecules detach from the sensor-side molecule.

According to various examples, it would be possible that the processor is configured to control the robotic actuator to dip the electrical sensor into the one or more wells in accordance with a predefined measurement script that implements the measurement protocol. I.e., it would be possible that the measurement script specifies a time sequence of wells into which the electrical sensor is being dipped in different iterations 3090 (e.g., depending on the content of each well), a time duration or generally timing of dipping an electrical sensor into the one or more wells, etc. Thus, the measurement script can include a time sequence of control instructions that specifies the movement of the robotic actuator. Accordingly, it would be possible that decision-making at box 3015 and/or decision-making at box 3025 is based on a predefined measurement script. Such predefined measurement script, accordingly, can specify in which wells of the MWP the electrical sensor is to be dipped and for how long, whether or not the electrical sensor is to be stirred in a given well, etc. The predefined measurement script can also define a duration of the dipping and/or a count of data readouts of the time sequence per well.

According the various examples, it would be possible that the predefined measurement script is parametrized based on at least one parameter. A value of the at least one parameter can be set based on at least one of the data readouts.

In other words, the measurement script can be interactive. Based on the data readouts of the measurement, certain properties of the remaining actions of the measurement can be adjusted. This is achieved by setting the value of the at least one parameter based on the at least one of the data readouts.

By implementing the measurement script in an (auto-)parametrized manner, it is, in particular, possible to avoid dead times. For instance, it would be possible to minimize a duration of phases of the measurement protocol (cf. TAB. 2) by monitoring a time evolution of the time sequence of data readouts in the respective phase. Throughput of samples can be increased.

A few examples of such parametrization of the measurement script will be explained below.

For instance, it would be possible that the measurement script defines the optional calibration phase or the dissociation, pre-detection or detection phase(cf. TAB. 2). The parameter of the parametrized measurement script that has an adjustable value could be the dwell time of the electrical sensor in the well filled with the reference liquid. The value of the dwell time than could be set based on a change rate of multiple data readouts while the electrical sensor is dipped into the respective well, and/or in absolute signal level of the data readouts while the electrical sensor is dipped into the respective well. such techniques are based on the finding that-after lowering the electrical sensor into the well filled with the reference liquid—it can be desirable to achieve a steady-state. In the steady-state, a reference value may be obtained from the respective data readouts and may be used to determine at least one property at box 3030.

In a further embodiment the predefined measurement script (185) defines a detection phase wherein the at least one processor (801) is configured to control the robotic actuator (102) to dip the electrical sensor (111) into the at least one well of the multi-well plate filled with the detection liquid during the detection phase. The at least one property to be measured comprises a concentration, the concentration being determined using a regression analysis of a predefined curve (45) to multiple data readouts (40) of the data readouts (40) of a time sequence of data readouts (40) acquired during the detection phase (59). The at least one parameter of the predefined measurement script accordingly comprises a time gate (51, 52) for the regression analysis and the value of the time gate (51, 52) is set based on a timing of said controlling of the robotic actuator (102) to dip the electrical sensor (111) into the at least one well (131-136) of the multi-well plate (130) filled with the detection liquid.

In a further embodiment the robotic actuator (102) is configured to engage a further electrical sensor (112) so that the electrical sensor (111) and the further electrical sensor (112) are arranged at an offset which corresponds to an offset between wells (133, 134) of the multi-well plate (130) and the at least one processor (801) is configured to control the further electrical sensor (112) to acquire one or more further time sequences of further data readouts when the further electrical sensor (112) is dipped into the at least one well being filled with the detection liquid of the multi-well plate (130) and the at least one processor (801) is configured to determine the at least one property using a reference baseline obtained from at least some of the further data readouts.

In further a preferred embodiment the at least one processor (801) is configured to control the robotic actuator (102) and the electrical sensor (111) in accordance with a predefined measurement script (185), the predefined measurement script (185) defines a preparation phase (57), the at least one processor (801) is configured to control the robotic actuator (102) to dip the electrical sensor (111) into a second one of the one or more wells (131-136) of the multi-well plate filled with a liquid comprising the first molecules during the preparation phase (57) and the at least one processor is configured to not dip the further electrical sensor (112) into any well (131-136) of the multi-well plate (130) filled with the liquid comprising the first molecules during the preparation phase (57).

The one property to be measured according to the present invention may be (i) binding kinetics of a binding between the second molecules and the target molecules, or of the third molecules and the second molecules or target molecules; (ii) a binding affinity of the binding between the second molecules and the target molecules or of the third molecules and the second molecules or target molecules, (iii) a concentration of the target molecules in the sample liquid, and (iv) a conformality structure of the target molecules.

As outlined above, the present invention further envisages a method for determining the presence and/or concentration of a target molecule in a sample. Said method comprises group of activities the provision of elements necessary for performed the method. Said group of activities comprises providing a robotic actuator (102) configured to engage an electrical sensor (111), the electrical sensor (111) comprising a sensitive region (180) that can be functionalized using first molecules of a first type, a platform (170) configured to retain a multi-well plate (130), and at least one processor (801) configured to control (3005, 3020) the robotic actuator (102) to dip the electrical sensor (111) into wells (131-136) of the multi-well plate (130). Further, the method requires the provision of a multi-well plate comprising at least one of the wells (131-136) being filled with an analyte liquid comprising second molecules of a second type, at least one further well (131-136) being filled with a liquid sample comprising a target molecule; at least one further well (131-136) being filled with a pre-detection liquid comprising third molecules of a third type; and at least one further well (131-136) being filled with a detection liquid comprising fourth molecules of a fourth type. The provision of one or more of the wells may vary depending on the overall setup of the method. For example, binding and association steps may be implemented in the preparation phase of the sensor or may be implemented via a subsequent association step by dipping the sensor in a further well. Accordingly, a corresponding dipping step may be present or absent depending on the necessity and logic of the interaction scheme for the detection of a target molecule or, as an alternative example, the testing or controlling of an antibody binding or interaction or the like.

The provision and presence of a well (131-136) within the system of the invention or used within the method of the invention may be required in accordance with the overall goal of the measurement approach and may thus be individually implemented or modified in line with the principles outlined herein.

In a further group of activities, the method comprises dipping said electrical sensor into at least one well (131-136) filled with an analyte liquid comprising second molecules of a second type, dipping said electrical sensor into at least one further well (131-136) filled with a sample liquid comprising a target molecule; dipping said electrical sensor into at least one further well (131-136) filled with a pre-detection liquid comprising third molecules of a third type; and dipping said electrical sensor into at least one further well (131-136) filled with a detection liquid comprising fourth molecules of a fourth type. The dipping steps may, as outlined above, depend on the presence of corresponding wells. For example, should there be no well filled with an analyte liquid, the dipping process may accordingly be modified. Further, certain steps may be repeated, individually skipped or performed for a predetermined period of time.

In a last group of activities a measurement procedure is performed. The present invention envisages a measuring an electrical signal in dependence of the electrochemical condition of the fourth molecules. This measurement typically detects an absolute current at the surface of the electrical sensor due to the interaction of the third molecule of the third type and the fourth molecule of the fourth type. Said measurement accordingly indicates the presence and/or concentration of the target molecule.

In particularly preferred embodiments the dipping steps with an electrical sensor comprising a sensitive region (180) that is optionally functionalized using first molecules of a first type are performed in specific order. This order may preferably have the following form:
(1) dipping said electrical sensor into at least one well (131-136) filled with an analyte liquid comprising second molecules; this step may, in certain embodiments, be skipped, e.g. in case the electrical sensor has been functionalized with an interactor of a target molecule and thus does not require the presence of a second molecule. In such a scenario the target molecule is typically modified in order to allow for an interaction with the functionalized electrical sensor.
(2) dipping said electrical sensor into at least one further well (131-136) filled with a sample liquid comprising a target molecule. In certain embodiments said target molecule may have been modified. Such a modification may performed outside of the multiwell plate or may be implemented as additional procedure within the current system and method, e.g. as additional modification steps.
(3) dipping said electrical sensor into at least one further well (131-136) filled with a pre-detection liquid comprising third molecules of a third type. The pre-detection liquid allows for a subsequent measurement of an absolute current in the presence of a fourth type of molecule; and
(4) dipping said electrical sensor into at least one further well (131-136) filled with a detection liquid comprising fourth molecules of a fourth type. This dipping steps leads to the generation of a current which can directly be detected at the surface of the electrical sensor.

In preferred embodiments, the method is for determining binding kinetics of a second molecule and a target molecule, or of the third molecule and the second molecule or target molecule. The necessary method steps correspond to those of the above described concentration measurement with the exception that the dipping and measurement activities are repeated. Such a repletion may be performed once, twice, 3 times, 4 times, 5 times or more often. Importantly, the repletion of the steps is performed after a predefined period of time. This period of time may be chosen according to the setup and conditions of the measurement and detection parameter and may accordingly vary. In certain embodiments, the period of time may be 30 sec, or 1, 2, 3, 4, 5, 10, 20, 30, 60 min or more. In further embodiments, the period of time may be 1 h to 5 h, or more. The period of time may further be changed during the repetition of steps or the performance of the method. It is particularly preferred that such time period is increased during the performance of the method. For example, if the first repetition is carried out after 1 min, a second repetition is carried out after 2, 3, 4, 5 mins or later etc. For example, the time period may be doubled, or multiplied with a specific factor.

In further preferred embodiments, the method is for determining binding affinity of the binding between the second molecules and the target molecules or of the third molecules and the second molecules or target molecules. The necessary method steps correspond to those of the above described concentration measurement with the exception that the dipping and measurement activities are repeated and that said dipping into at least one further well (131-136) is performed in a well which comprises an increased or, alternatively, decreased concentration of the pre-detection liquid comprising third molecules of a third type in comparison to the previously used well. For example, said concentration may be increased by a factor 2, 3, 4, 5, 6, 7, 8, 9, 10, 20, 40, 100, 1000, 10 000, 100 000 or more, or any other value in between the mentioned values in in comparison to the previously used well.

In further embodiments said concentration may be decreased by a factor 2, 3, 4, 5, 6, 7, 8, 9, 10, 20, 40, 100, 1000, 10 000, 100 000 or more, or any other value in between the mentioned values in in comparison to the previously used well.

In further preferred embodiments, the method is for determining the conformality structure of the target molecules. The necessary method steps correspond to those of the above described concentration measurement with the exception that dipping of said electrical sensor into at least one further well (131-136) is performed in a well which comprises a liquid with a pH differing from the pH of the liquid in the previously used well.

In particularly preferred embodiments the first molecule may, for example, be an avidin-like molecule such as a streptavidin or avidin or a functionally or structurally similar variant. For example, the variant may be tamavidin, e.g. tamavidin 1 or tamavidin 2, shwanavidin, rhizavidin, bradavidin, burkavidin, zebavidin, xenavidin or strongavidin. The present invention additionally provides for the use of modified avidins, e.g. the use of proteins which have been genetically optimized, whose sequences have been adapted or which have been altered post-translationally, e.g. via chemical modifications of amino acid side groups. In further embodiment the first molecule may, for example, be a hydrophobic molecule such as an alkane, lipid, hydrophobin or the like. In further embodiments the first molecule may, for example, be a molecule which is capable of interacting with an immunoglobulin such as protein A, protein G, protein L or jacalin, which bind to the Fc region of an immunoglobulin. In further embodiments, the first molecule may be a molecule with a free carboxylic acid functional group, allowing for crosslinking to other molecules containing amine functional groups through the formation of a peptide bond.

In particularly preferred embodiments the second molecule may generally be an molecule which is capable of interacting with or binding to a first molecule as defined above. For example, the second molecule may be an immunoglobulin molecule such as an antibody or a variant thereof, which is capable of binding a first molecule, e.g. protein A. Alternatively, the second molecule may be protein A, which may be connected to a linker molecule provided as first molecule as defined above. In a further embodiment, the second molecule of a second type may be capable of interacting with an avidin or streptavidin molecule which was previously connected to the sensor as first molecule. Such an interactor of avidin/streptavidin may, for example, be a biotinylated compound, e.g. a protein having been biotinylated. Should the second molecule be, for example, a functionalized protein or compound, there may be no target molecule present. Accordingly, the protein component of the functionalized protein may constitute the target protein to be measured.

In particularly preferred embodiments the target molecule may generally be a molecule which is capable of interacting with or binding to said second molecule as defined above. Typically, the target molecule derivable from a sample. Examples of such target molecules include small organic molecules, proteins, e.g. an interleukin such as IL-6 or any other polypeptide, or an antibody to be analyzed, such as an antibody produced in bioreactor etc. In certain embodiments said third molecule may not be provided in a well since the presence of a functionalized second molecule renders its presence superfluous.

In particularly preferred embodiments the third molecule of a third type may generally be an molecule which is capable of interacting with or binding to a target molecule or a second molecule as defined above. Said third molecule is further part of a pre-detection liquid and allows for a subsequent detection reaction of the presence of a target molecule or second type molecule. For example, the third molecule may be a secondary antibody binding to a primary antibody, or binding to an antibody to be detected from a bioreactor. Said third molecule is typically functionalized with an enzymatic activity which is capable of producing an absolute current. Examples of such enzymatic activities include horseradish peroxidase (HRP), alkaline phosphatase (ALP), glucose peroxidase, alpha-glucosidase, DNA polymerase.

In particularly preferred embodiments the fourth molecule of a fourth type is a molecule which interacts with the third molecule and thereby produces an absolute current. This molecule is part of the detection liquid. An example of said molecule is 3,3′,5,5′-tetramethylbenzidine (TMB). Further examples include pyridoxal 5′-phosphate, glucose, dissaccharide maltose or DNA.

According to certain embodiments of the invention the method may be performed with a first molecule as defined herein, a target molecule as defined herein, a third molecule as defined herein and a fourth molecule as defined herein. A system as defined herein may accordingly comprise correspondingly filled wells.

In further embodiments, the present method may be performed with a first molecule as defined herein, a second molecule as defined herein, a target molecule as defined herein and a fourth molecule as defined herein. A system as defined herein may accordingly comprise correspondingly filled wells.

In further embodiments, the present method implements ELISA detection approaches as known to the skilled person. For example, the system according to the present invention or the presently claimed methods may be used for the performance of a direct ELISA detection, an indirect ELISA detection, a sandwich ELISA detection or other ELISA detections or immunological detection formats known to the skilled person.

The following examples and figures are provided for illustrative purposes. It is thus understood that the example and figures are not to be construed as limiting. The skilled person in the art will clearly be able to envisage further modifications of the principles laid out herein.

### EXAMPLES

### Example 1

The experiment was carried out with 3 sensors to measure the concentration differences in the analyte.

During this experiment biotinylated IL6 was used as analyte.

The sensor was coated with analyte: Well A1-C1 100 μL of 1 ng/mL-500 ng/mL biotinylated IL6.

This was followed by a washing step: Well A2-C2 PBS wash buffer

This was followed by a interaction with an target: Well A3-C3 Anti-IL6 Antibody. Concentration 1 nM, 10 nM, 100 nM.

This was followed by a further washing step: Well A2-C2 PBS wash buffer.

This was followed by a pre-detection step: well A4-C4 Secondary Antibody with HRP tag. Concentration 10 nM

This was followed by a further washing step: Well A2-C2 PBS wash buffer.

Finally, a detection step was performed: Well A5-C5 TMB substrate. Measurement was performed immediately after dipping

The results of the experiment are shown in FIG. 7.

1. A system, comprising:
a robotic actuator configured to engage an electrical sensor, the electrical sensor comprising a sensitive region that can be functionalized using first molecules of a first type,
a platform configured to retain a multi-well plate, and
at least one processor configured to control the robotic actuator to dip the electrical sensor into wells of the multi-well plate, wherein at least one of the wells is filled with an analyte liquid comprising second molecules of a second type,
wherein at least one further well is filled with a sample liquid comprising a target molecule;
wherein at least one further well is filled with a pre-detection liquid comprising third molecules of a third type;
wherein at least one further well is filled with a detection liquid comprising fourth molecules of a fourth type;
wherein the at least one processor is configured to control the electrical sensor to acquire one or more time sequences of data readouts when the electrical sensor is dipped into the at least one well being filled with the detection liquid,
wherein the at least one processor is configured to determine at least one property of the target molecule or of molecules interacting with it based on at least some of the data readouts of the one or more time sequences of data readouts.

2. The system of claim 1,
wherein the at least one processor is configured to determine the at least one property based on a timing of said controlling of the robotic actuator to dip the electrical sensor into one or more wells of the multi-well plate.

3. The system of claim 1, wherein the at least one of the wells being filled with an analyte liquid comprising second molecules of a second type is not present in the system.

4. The system of claim 1,
wherein the at least one processor is configured to control the robotic actuator and the electrical sensor in accordance with a predefined measurement script,
wherein the predefined measurement script is parameterized based on at least one parameter, a value of the at least one parameter being set based on at least one of the data readouts of the one or more time sequences of data readouts.

5. The system of claim 4,
wherein the predefined measurement script defines a detection phase,
wherein the at least one processor is configured to control the robotic actuator to dip the electrical sensor into the at least one well of the multi-well plate filled with the detection liquid during the detection phase,
wherein the at least one property comprises a concentration, the concentration being determined using a regression analysis of a predefined curve to multiple data readouts of the data readouts of a time sequence of data readouts acquired during the detection phase wherein the at least one parameter of the predefined measurement script comprises a time gate for the regression analysis,
wherein a value of the time gate is set based on a timing of said controlling of the robotic actuator to dip the electrical sensor into the at least one well of the multi-well plate filled with the detection liquid.

6. The system of claim 1,
wherein the robotic actuator is configured to engage a further electrical sensor so that the electrical sensor and the further electrical sensor are arranged at an offset which corresponds to an offset between wells of the multi-well plate,
wherein the at least one processor is configured to control the further electrical sensor to acquire one or more further time sequences of further data readouts when the further electrical sensor is dipped into the at least one well being filled with the detection liquid of the multi-well plate,
wherein the at least one processor is configured to determine the at least one property using a reference baseline obtained from at least some of the further data readouts.

7. The system of claim 6,
wherein the at least one processor is configured to control the robotic actuator and the electrical sensor in accordance with a predefined measurement script,
wherein the predefined measurement script defines a preparation phase,
wherein the at least one processor is configured to control the robotic actuator to dip the electrical sensor into a second one of the one or more wells of the multi-well plate filled with a liquid comprising the first molecules during the preparation phase,
wherein the at least one processor is configured to not dip the further electrical sensor into any well of the multi-well plate filled with the liquid comprising the first molecules during the preparation phase.

8. The system of claim 1,
wherein the at least one processor is configured to control at least one of the robotic actuator or a motor attached to the platform to relative move the electrical sensor with respect to and within the at least one of the one or more wells when the electrical sensor is dipped into each one of the at least one of the one or more wells.

9. The system of claim 1,
wherein the at least one property comprises at least one of
(i) binding kinetics of a binding between the second molecules and the target molecules, or of the third molecules and the second molecules or target molecules;
(ii) a binding affinity of the binding between the second molecules and the target molecules or of the third molecules and the second molecules or target molecules,
(iii) a concentration of the target molecules in the sample liquid, or
(iv) a conformality structure of the target molecules.

10. A method for determining the presence and/or concentration of a target molecule in a sample, comprising:
(A) providing
a robotic actuator configured to engage an electrical sensor, the electrical sensor comprising a sensitive region that can be functionalized using first molecules of a first type,
a platform configured to retain a multi-well plate, and
at least one processor configured to control the robotic actuator to dip the electrical sensor into wells of the multi-well plate-,
a multi-well plate comprising at least one of the wells being filled with an analyte liquid comprising second molecules of a second type, at least one further well being filled with a liquid sample comprising a target molecule; at least one further well being filled with a pre-detection liquid comprising third molecules of a third type; and at least one further well being filled with a detection liquid comprising fourth molecules of a fourth type;
(B) dipping said electrical sensor into at least one well filled with an analyte liquid comprising second molecules of a second type,
dipping said electrical sensor into at least one further well filled with a sample liquid comprising a target molecule;
dipping said electrical sensor into at least one further well filled with a pre-detection liquid comprising third molecules of a third type; and
dipping said electrical sensor into at least one further well filled with a detection liquid comprising fourth molecules of a fourth type;
(C) measuring an electrical signal in dependence of the electrochemical condition of the fourth molecules, indicating the presence and/or concentration of the target molecule.

11. The method of claim 10, wherein the dipping steps are performed in the following order:
(1) dipping said electrical sensor into at least one well filled with an analyte liquid comprising second molecules,
(2) dipping said electrical sensor into at least one further well filled with a sample liquid comprising a target molecule,
(3) dipping said electrical sensor into at least one further well filled with a pre-detection liquid comprising third molecules of a third type; and
(4) dipping said electrical sensor into at least one further well filled with a detection liquid comprising fourth molecules of a fourth type.

12. The method of claim 10, wherein said method is for determining the binding kinetics of a binding between the second molecules and the target molecules, or of the third molecules and the second molecules or target molecules, comprising a repetition of step (B) and (C) after a predefined period of time, preferably after 30 sec, 1, 2, 3, 4, 5, 10, 20, 30, 60 min to 5 h, wherein said repetition is a 1×, 2×, 3×, 4×, 5× or multiple repetition.

13. The method of claim 10, wherein said method is for determining the binding affinity of the binding between the second molecules and the target molecules or of the third molecules and the second molecules or target molecules, comprising a repetition of step (B) and (C) wherein after each execution or repetition of steps (B) and (C) the dipping of said electrical sensor into at least one further well is performed in a well which comprises an increased or decreased concentration of the pre-detection liquid comprising third molecules of a third type in comparison to the previously used well.

14. The method of claim 13, wherein said concentration is increased or decreased by a factor 2, 3, 4, 5, 6, 7, 8, 9, 10 or more in comparison to the previously used well.

15. The method of claim 13, wherein after each execution or repetition of steps (B) and (C) said electrical sensor is in step (D) dipped into at least one well filled with a dissociation liquid capable of dissociating the bond between the second molecule of a second type and the third molecule of a third type.

16. The method of claim 13, wherein after each dipping step the sensor is additionally dipped into at least one well filled with a rinsing liquid.

17. The method of claim 10, wherein said method is for determining the conformality structure of the target molecules, comprising a repetition of step (B) and (C) wherein after each execution or repetition of steps (B) and (C) the dipping of said electrical sensor into at least one further well is performed in a well which comprises a liquid with a pH differing from the pH of the liquid in the previously used well.

18. (canceled)

19. (canceled)

20. (canceled)

21. The method of claim 10, wherein said method is performed with a system comprising:
a robotic actuator configured to engage an electrical sensor, the electrical sensor comprising a sensitive region that can be functionalized using first molecules of a first type,
a platform configured to retain a multi-well plate, and
at least one processor configured to control the robotic actuator to dip the electrical sensor into wells of the multi-well plate, wherein at least one of the wells is filled with an analyte liquid comprising second molecules of a second type,
wherein at least one further well is filled with a sample liquid comprising a target molecule;
wherein at least one further well is filled with a pre-detection liquid comprising third molecules of a third type;
wherein at least one further well is filled with a detection liquid comprising fourth molecules of a fourth type;
wherein the at least one processor is configured to control the electrical sensor to acquire one or more time sequences of data readouts when the electrical sensor is dipped into the at least one well being filled with the detection liquid,
wherein the at least one processor is configured to determine at least one property of the target molecules or of molecules interacting with it based on at least some of the data readouts of the one or more time sequence of data readouts.

22. The system of claim 1, wherein said second molecule is an antibody or a specifically binding non-immunoglobulin, said third molecule is an antibody or a specifically binding non-immunoglobulin which is conjugated to an enzymatic activity, preferably HRP, and/or said fourth molecule is an electrochemically sensitive molecule whose electrochemical condition can be changed by the enzymatic activity.

23. The method of claim 10, wherein said second molecule is an antibody or a specifically binding non-immunoglobulin, said third molecule is an antibody or a specifically binding non-immunoglobulin which is conjugated to an enzymatic activity, preferably HRP, and/or said fourth molecule is an electrochemically sensitive molecule whose electrochemical condition can be changed by the enzymatic activity.
